(19) United States
(12) Patent Application Publication
(10) Pub. No.: US 2012/0270735 A1
(43) Pub. Date: Oct. 25, 2012

(54) PREPARATION OF PGPR BIOFORMULATION FOR BIOLOGICAL CONTROL OF RED ROOT ROT DISEASE IN TEA PLANTS

(76) Inventors: MANJUKARUNAMBIKA KOLANDASAMY, NAMAKKAL DISTRICT (IN); P. PONMURUGAN PONNUSAMY, NAMAKKAL DISTRICT (IN)

(21) Appl. No.: 13/092,178

(22) Filed: Apr. 22, 2011

(51) Int. Cl. C05F 15/00; A01P 3/00; C12N 11/14; C12N 1/20

(52) U.S. Cl. 504/102; 435/253.3; 435/176

(57) ABSTRACT

According to the present invention, a process is designed for preparation of PGPR bioformulation having superior viability by using Pseudomonas fluorescens VP5 isolated from tea rhizosphere. The invention is primarily designed to develop a PGPR formulation process for biocontrol of red root rot disease. This strain is useful as plant growth promoting rhizobacterium (PGPR) and biocontrol agent which can be formulated to produce antibiotic compounds that is active against the phytopathogen Poria hypolateritia. The process involves culturing of Pseudomonas fluorescens in a growth medium amended with colloidal chitin. The resulting mixture is then mixed with talc based carrier. The carrier with immobilized bioagent along with combination of vermicompost and farmyard manure is selected as efficient bioformulation for controlling root pathogen.

### FIELD OF INVENTION

The present invention relates to novel biological composition with an indigenous bacterial strain, an organic chitinolytic material, plant growth promoting materials and a carrier material for delivery of Pseudomonas fluorescens. The amendment of organic material is for both plant growth inducer as well as defense mechanism in plants and for controlling the root pathogenic fungus. More specifically the present invention relates to a specific bacterium Pseudomonas fluorescens having antagonistic property against tea fungal pathogen Poria hypolateritia. This strain grows well in the rhizosphere of tea plants to sufficiently reveal the antagonistic property against the root pathogen and is being able to promote growth of tea plants indirectly. In addition, the invention relates to growth improvements of plant roots. In particular, the invention relates to preparation of improved biological compositions which are efficient to increase the growth rate of plant and to develop induced systemic disease immunity in plants to control soil pathogen. The process of present invention further relates to evolution of biological composition comprising of organic compound disaccharide amended in the growth medium for extended shelf life and population density of bacterial strain in carrier material which combat fungal pathogen as efficient biocontrol agent after delivery of the bacteria to soil. It also relates to bacterial strain present in the compositions which have been found to inhibit the growth of fungal tea pathogen by the combined formulation to produce antifungal metabolites such as antibiotic compounds.

### PRIOR ART/BACKGROUND OF THE INVENTION

Plants exert defense mechanism against the pathogen by systemic acquired resistance (SAR) or induced systemic resistance (ISR). The plant growth-promoting rhizobacterium (PGPR) suppresses pathogen in plants by eliciting ISR. Naturally occurring pathogens suppressiveness has been reported for several agricultural systems (Kluepfel et al., 1993). The tea plants belong to the family Camelliaceae that contains 82 species of the genus Camellia. The tea plant is an evergreen of the Camellia family that is native to China, Tibet and Northern India. The small leaf variety known as Camellia sinensis (L) O. Kuntze, thrives in the cool, high mountaneous regions. Perennial habit of the tea plant, peculiar culture conditions and warm humid climate of the tea growing areas are highly conducive for disease development. The majority of diseases in tea are of fungal origin affecting the parts of leaf, stem and roots of tea bush. Among that, incidence of red root disease caused by Poria hypolateritia and its severity is found to be high in tea plants at Valparai.

Plant-associated microorganisms have been extensively examined for their roles in natural and induced suppressiveness of soilborne diseases. Among the many groups of such organisms are root-associated bacteria, which generally represent a subset of soil bacteria. Rhizobacteria which exert a beneficial effect on the plant being colonized are termed as PGPR. Among them Pseudomonas fluorescens is one of the well studied bacteria for its root colonization in plants. It benefits the host by causing plant growth promotion and biological disease control. Efforts to select and apply PGPR for control of specific soil borne fungal pathogens have been studied extensively. Mixtures of PGPR strains showed a higher level of disease protection in case of cucumber diseases (Raupach and Kloepper, 2000). Biological agents those that colonize the plant, usually roots, have been shown to increase the plant growth or control plant diseases. Selected PGPR strains belonging to Gram-negative genera Pseudomonas upon soil drench treatment to plant root systems reduce the incidence of distally infecting pathogens.

Chitin (C₈H₁₃O₅N)n is a long-chain polymer of a N-acetylglucosamine, a derivative of glucose is a structural component of some fungi. It is the main component present in the cell walls of fungi. The breakdown of this polymer by chitinases can cause lysis of fungi. Similar to chitin amendment in bioformulation, betaine is added to biological composition to increase survival of the microorganism for the preparation of bacterial agricultural products according to Kosanke et al., U.S. Pat. No. 5,695,541. In the soil, chitinases are produced by many plants and also released as part of their defense mechanism against various pathogens (Punja and Zhang, 1993). Benhamou et al. (1994) have shown that chitosan, the deacetylated derivative of chitin, induces systemic plant resistance against Fusarium oxysporum f. sp. radicis-lycopersici in tomato when applied as a seed treatment or soil amendment. This suggests that plant defense mechanisms might contribute to the overall pathogenic fungal suppression. Most recent studies point out that chitosan is a good inducer, fertilizer and act as ecologically friendly biopesticide for defense mechanisms in plants, which gives better yield, life expectancy and hence profoundly used in agriculture. Naturally occurring pathogen suppressiveness can also be induced by organic amendments including chitin (Kokalis-Burelle et al., 2002).

Vermicompost is composed of organic materials derived from plant and animal matter that has been decomposed largely through aerobic decomposition. Compost is rich in nutrients. It is generally recommended as an additive to soil for addition of vital humus or humic acids. It act as soil conditioner, fertilizer and as a natural pesticide for soil. It being primarily mixed with soil which provides a rich growing medium and its porous absorbent material that holds moisture and soluble minerals, providing the support and nutrients in which plants can flourish.

Farmyard manure (FYM) is an excellent source of plant nutrients. Approximately 70-80% of the nitrogen, 60-85% of the phosphorus and 80-90% of the potassium and trace elements present in manure and improve soil fertility. The availability or efficiency of manure utilization by a crop is determined by the method of application, time to incorporation and the rate of manure decomposition by microorganisms in soil. The availability of NPK is directly available to the plants by the application of manure.

### CONTRIBUTION OF THE INVENTION

An adequate strategy has been developed that provides a satisfactory, simple, economic, and reliable process for biological composition preparation that is suitable to treat tea plants from a very early stage to high leaf yield fast-growing plants which have an induced systemic resistance against root pathogens. Ultimately further alternatives to chemical fungicides for control of plant diseases are needed. Reducing the pressure and dependence of control of plant diseases on chemical fungicides solutions is highly desired in this process invention.

### OBJECTIVES OF THE INVENTION

The key object of the present invention is to develop a standard process for the preparation of novel PGPR bioformulation containing Pseudomonas fluorescens for the biological control of red root rot disease caused by Poria hypolateritia in tea plants.

Another object of the present invention is to prepare a typical bioformulation that enhance the activity, viability and mass multiplication of P. fluorescens during different storage conditions as well as during field application to enhance the population density and survival rate.

Yet another object of the present study is organic amendment of chitosan in talc formulation for effective biological control of a phytopathogen P. hypolateritia.

Still another object of the present invention is to prepare PGPR bioformulation which has growth medium for sustainable mass multiplication and a carrier material for immobilizing and delivery P. fluorescens to tea soil.

Additional object of the present investigation is to combine the resulting bioformulation with vermicompost and farmyard manure as additive soil conditioners and for rapid mass multiplication.

### DESCRIPTION OF INVENTION

The present invention broadly relates to the PGPR formulation process with innovative biological compositions, specifically to preparation methods for biocontrol of root disease. According to the present invention a new strategy of biological composition preparation for effective biocontrol of red root rot disease against Poria hypolateritia has been developed. One aspect of the present invention provides an improved process for biotalc formulation product with increased shelf life of bacteria for long time storage and upon revitalization.

In the present invention Pseudomonas fluorescens was inoculated in modified King's B liquid medium which includes the amendment of sucrose for increased population and shelf life of Pseudomonas fluorescens in talc formulation.

The invention embraces the preparation of antifungal compositions which comprises indigenous live bacterium Pseudomonas fluorescens VP5 and its metabolites, a natural active ingredient organic biocontrol elicitor such as chitin/chitosan mixed, talc powder, carboxy methyl cellulose, vermicompost and farm yard manure.

The organic amendment is also known as a chitinolytic component as it exhibits fungicidal control activity. The amount of chitinolytic component present in the composition is sufficient to cause a chitinolytic effect and it may range from 1% to 10.0%. A preferred embodiment of the present invention is where the composition consist of approximately 2.5% of the chitinolytic component. Examples of the chitinolytic component include chitin, chitin flakes and chitosan. Preferably, the organic amendment is a glucose polysaccharide. Such precursors are organic natural compounds like pine bark, crab or shrimp shells, soybean meal, cotton seed meal and casein.

Further the process invention relates to a new synergistic method of using composition of a chitinolytic element and bacterial elements in the bioformulation preparation for biocontrol of red root disease. It is one important aspect of the present invention that a potential strain of Pseudomonas fluorescens are provided in this bioformulation is found to have antifungal activity that is active against root pathogenic fungi Poria hypolateritia. Vermicompost is for supplying plant nutrients, carboxy methyl cellulose as adhesive and farm yard manure which creates a synergy in plant growth and disease resistance.

This biological composition is important for several reasons. First, Poria hypolateritia is particularly a pernicious plant pathogen. It mainly affects the root parts of tea plants. Moreover, there is no effective biofungicide treatment available for the protection of crops from this pathogen. Therefore, the use of this novel biological composition prevents this fungal infection in tea plants. This may provide an environmentally safe and effective method for control of this pathogen. Also the present invention in mixtures in order to improve the biocontrol capabilities against P. hypolateritia is also part of the present invention.

In the present invention preferred methods of applying an agrochemical composition which contains at least one of the antifungal metabolites produced by the bacterial strains is soil application. The number of applications and the rate of application depend on the intensity of infestation by the corresponding pathogen. However, the active organic ingredients can also penetrate the plant through the roots via the soil by impregnating the locus of the plant with a liquid composition or by applying the compounds in solid granular form to the soil.

### Example-1

The following are the composition of modified King's B liquid medium used for growth of Pseudomonas fluorescens VP5

| Modified King's B medium | gms/litre |
|---|---|
| Proteose peptone # 3 | 20 g |
| K₂HPO₄ | 1.5 g |
| MgSO₄•7H₂O | 1.5 g |
| Sucrose | 100 g |
| Colloidal chitin | 2.5% |

### Example-2

TABLE 2
Morphological, cultural and biochemical characteristic
of rhizobacteria

| | Pseudomonas |
|---|---|
| Characteristics | fluorescens VP5 |
| Gram's staining | Negative |
| Fluorescence In UV | Positive |
| Motility test | Positive |
| Shape | Rod |
| Endospore Formation | No spore |
| Indole test | Negative |
| Methyl red test | Negative |
| Voges proskauer test | Negative |
| Catalase test | Positive |
| Oxidase test | Positive |
| Citrate test | Positive |
| Urease test | Positive |
| Triple sugar iron test | Negative |
| O.F. media | Positive |
| H₂S production | Negative |
| Nitrate reduction | Positive |
| Starch hydrolysis | Positive |
| Gelatin hydrolysis | Positive |
| Growth at 4° C. | Positive |
| Growth at 44° C. | Negative |

### Example-3

TABLE 2
Utilization of carbon sources and nitrogen sources by
Pseudomonas fluorescens VP5

| | |
|---|---|
| Carbon sources | Nitrogen sources |

| | | | |
|---|---|---|---|
| Glucose | + | Peptone | + |
| Fructose | − | Yeast extract | + |
| Sucrose | + | Urea | − |
| Lactose | − | Ammonium chloride | − |
| Glycerol | + | Ammonium sulphate | + |
| Olive oil | + | Ammonium nitrate | + |

### Example-4

### Detection of Phosphate Solubilization by Pseudomonas fluorescens VP5 by Plate Assay

The following are the composition of modified Pikovskaya's solid medium contained in g/L of distilled water used for P-Solubilization of Pseudomonas fluorescens VP5

Yeast extract—0.50

### Dextrose—10.00

### Ca₃(PO₄)₂—5.00

### MgSO₄.7H₂O—0.25

### Agar—15.0

TABLE 3
Phosphate solubilization in plate assay

| | Diameter of halo zone around the colony (cm) | | | | | | |
|---|---|---|---|---|---|---|---|
| Test strain | 1 | 2 | 3 | 4 | 5 | 6 | 7 |
| P. fluorescens VP5 | 0.5 | 1.3 | 1.5 | 2.1 | 2.7 | 3.4 | 4.1 |

### Example-5

### Antibiosis

This was determined by slight modification cellophane over lay technique of was performed. A 48 hrs old culture of Pseudomonas fluorescens as bacterial antagonist was grown in King's B broth. This culture was smeared over the entire surface of the cellophane. The Petri plates containing malt extract medium over laid with smeared cellophane were incubated in dark at 25° C. for 48 hrs after which the cellophane sheets were removed. A single 6 mm mycelial plug of Poria hypolateritia from 1 week old culture was placed over the MEA plates. The plates were incubated at 25° C. in the dark for 10 days. The cellophane overlays smeared with sterile distilled water served as the control. The area covered by Poria hypolateritia was recorded in terms of diameter of pathogen in the petridishes. Three replicates were used throughout the study for all experiments including the controls. The percentage inhibitions growth of treated were calculated using the formula P═(C−I)/C×100 where P is the percentage growth of growth inhibition, C is the average radial growth of the pathogen in control plates and I is the average radial growth of the pathogen in treated plates.

TABLE 4
Antifungal activity of Pseudomonas fluorescens by
antibiosis method

| | Linear growth of Poria hypolateritia in mm | | |
|---|---|---|---|
| Days | Control | Treated | Inhibition over control (%) |
| 3 | 9.7 | 5.3 | 45.3 |
| 6 | 18.0 | 9.7 | 46.1 |
| 9 | 24.3 | 13.0 | 46.5 |
| 12 | 33.7 | 15.7 | 53.4 |
| 15 | 37.0 | 16.3 | 55.9 |
| 18 | 41.3 | 17.0 | 58.8 |
| 21 | 45.0 | 17.0 | 62.2 |

Pathogen alone was inoculated in control plates. Pathogen and antagonist was inoculated in treated plates.

### Example-6

### Preparation of Bacterial Suspensions

The P. fluorescens cells were harvested and centrifuged at 6000 rpm for 15 min and resuspended in phosphate buffer (0.01 M, pH 7.0). The concentration was adjusted using a spectrophotometer to approximately 10⁸ cfu/ml (OD₆₂₀=0.3) and used as bacterial inoculum (Thompson, 1996). These isolates were kept at −80.0 in 44% glycerol and cells from stocks were first grown on KB medium. The inoculum was produced by transferring one loopful from that culture to 100 ml of modified KB broth with 2.5% of chitin in a 250 ml of Erlenmeyer flask and incubating at room temperature (28±2′C) on a shaker at 150 rpm for 48 hrs.

### Example 7

### Selection of Carrier for Bioformulation of P. fluorescens

Different carrier materials were tested to determine the increased shelf life, survival rate of bacteria and long time storage with maintenance of population density of P. fluorescens. The PGPR was challenged with various industrial and agricultural waste products such as peat, zeolite, alginate, press mud, commercial talc powder, coconut coir pith, mushroom waste, cow dung, lignite powder, flyash, saw dust, sugarcane molasses, saw-dust-soil molasses, rice bran and vermicompost. Among them talc was selected as carrier material for immobilizing the bacteria. It has very low moisture equilibrium, relative hydrophobicity, chemical inertness that enables longer storage periods.

### Example 8

### Preparation of Mineral Carrier

The powdered mineral of talc was chosen as carrier for formulation. The carrier materials were steam sterilized at 140 Kpa for 30 min and dried aseptically in plastic trays for 12 hrs at 50° C. before using.

### Example 9

### Development of Talc Based Formulations of Pseudomonas fluorescens

One loopful of individual Pseudomonas fluorescens was inoculated in to modified King's B broth amended and incubated on shaker incubator at 150 rpm for 48 h at room temperature (28±2° C.). After 48 hrs of incubation the broth containing 9×10⁸ cfu/ml was used for the preparation of talc based formulations. To 400 ml of bacterial suspension, a mixture of 1 kg of a purified talc powder, 15 g of calcium carbonate (adjusted to neutral pH), 10 g of carboxy methyl cellulose (CMC adhesive) was prepared under sterile conditions following the method described by Vidhyasekaran and Muthuamilan (1995). The product was shade dried to reduce the moisture content (less than 20%) and then packed in to polypropylene bags and sealed. The population density of bacteria was varied between 2.5-3.5×10⁸ cfu/g of talc powder at the time of application.

### Example 10

### Shelf Life of the Talc Formulation of Biocontrol Agents

Periodic sampling of talc was to determine the shelf life and population density of P. fluorescens VP5. The shelf life of the talc-based formulations of biocontrol agents stored for 6 months at room temperature (28±2° C.) was studied by a serial dilution technique. One gram of the sample drawn from each formulation periodically at 30, 60, 90, 120, 150 and 180 days of storage was mixed with 10 ml of sterile distilled water. From this serial dilutions were made. One ml aliquot of each dilution was pipetted out into sterilised Petri plates containing King's B broth supplemented with rifampicin 200 μg/ml, penicillin 150 vg/ml, tetracycline 50 vg/ml kanamycin 100 vg/ml was added and incubated at room temperature (28±2° C.). The number of colony forming units of bacteria was counted 3 days after plating and expressed as the number of CFU/g of formulation. The shelf life of talc was determined to recognize the viability and concentration of active cells present in the mixture.

TABLE 5
Enumeration of population density of P. fluorescens in talc carrier

| No of | Enumeration of population |
|---|---|
| months | density of VP5 in talc |
| 1 | 3.5 × 10⁸ |
| 2 | 2.7 × 10⁸ |
| 3 | 1.4 × 10⁷ |
| 4 | 7.0 × 10⁶ |
| 5 | 5.2 × 10⁶ |
| 6 | 2.8 × 10⁶ |

### Example 11

### Vermicompost and Farmyard Manure (FYM) Based Formulation for Mass Multiplication

The talc formulation was mixed with farm yard manure and vermicompost in the ratio of 1:100 and incubated for one week. About half kg of is added to each plant. In the following table the growth and population density of root colonizing P. fluorescens VP5 strain was calculated by using antibiotic plasmid resistance method. This bioformulation was applied in tea soil for every six months by using different combination of treatments (Table 6).

TABLE 6
Establishment of Pseudomonas fluorescens in tea rhizosphere

| | Population density of P. fluorescens at | | | |
|---|---|---|---|---|
| | six month interval | | | |
| | First | Second | Third | Fourth |
| Treatment details | six month | six month | six month | six month |
| Vermicompost + Talc | 2.1 × 10³ | 5.3 × 10³ | 1.6 × 10⁴ | 3.7 × 10⁴ |
| (1:100) | | | | |
| Farmyard manure + Talc | 8.0 × 10³ | 3.8 × 10⁴ | 6.3 × 10⁴ | 1.4 × 10⁵ |
| (1:100) | | | | |
| Vermicompost + | 2.6 × 10⁴ | 7.4 × 10⁴ | 1.9 × 10⁵ | 2.3 × 10⁵ |
| Farmyard manure + | | | | |
| Talc (1:50:50) | | | | |

What claimed is:

1. A process for making biological composition consisting of pure strain of root colonizing PGPR, Pseudomonas fluorescens is selected from tea rhizosphere that have high biocontrol activity and effectively inhibited a soil-borne tea root pathogen Poria hypolateritia.

2. A biological composition according to claim 1, comprising pure culture of P. fluorescens grown in modified King's B broth amended with sucrose containing population density of cells 9×10⁸ cfu/ml is used for the preparation of carrier based formulation.

3. The process as claimed in claim 2, the King's B broth additionally includes amendment of organic ingredient chitin/chitosan, a natural active biocontrol elicitor in an amount of 2.5% which does not detrimentally affect the function of the PGPR.

4. The process as claimed in claim 2, the pure culture of P. fluorescens is prepared in King's B broth supplemented with further addition of antibiotics from the group consisting of rifampicin, tetracycline, kanamycin and penicillin.

5. The process of biological composition as claimed in claim 2, the pure culture of P. fluorescens is mixed with suitable carrier that has increased shelf life, for long survival of bacteria with maintenance of population density, very low moisture equilibrium and chemical inertness that enable longer storage periods.

6. A biological composition according to claim 5, the biocontrol agent P. fluorescens is then immobilized using talc powder as inorganic culture carrier.

7. A biological composition according to claim 6, further the biotalc formulation mixed with vermicompost and farm yard manure as additive soil conditioner and for mass multiplication which is applied in tea soil for every six months.
